SEQUENCE LISTING (74 sequences)

SEQ ID NO: 1 - protein, 441 aa, Artificial Sequence
  Tau
MAEPRQEFEVMEDHAGTYGLGDRKDQGGYTMHQDQEGDTDAGLKESPLQTPTEDGSEEPGSETSDAKSTPTAEDVTAPLVDEGAPGKQAAAQPHTEIPEGTTAEEAGIGDTPSLEDEAAGHVTQARMVSKSKDGTGSDDKKAKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKNVKSKIGSTENLKHQPGGGKVQIINKKLDLSNVQSKCGSKDNIKHVPGGGSVQIVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSSTGSIDMVDSPQLATLADEVSASLAKQGL

SEQ ID NO: 2 - protein, 16 aa, Artificial
  D1.2 LC CDR1
RSSQSLVHSNGNTYLH

SEQ ID NO: 3 - protein, 7 aa, artificial
  D1.2 LC CDR2
KVSNRFS

SEQ ID NO: 4 - protein, 7 aa, artificial
  D1.2 LC CDR3
SQSTHVP

SEQ ID NO: 5 - protein, 219 aa, artificial
  D1.2 VL
DVMMTQTPLSLPVSLGDQASISCRSSQSLVHSNGNTYLHWHLQKPGQSPKFLIYKVSNRFSGVPDRFSGSGSGTDFTLKISRVEAEDLGVYFCSQSTHVPFTFGSGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 6 - protein, 13 aa, artificial
  D1.2 HC CDR1
KASGNTFTDYEIH

SEQ ID NO: 7 - protein, 17 aa, artificial
  D1.2 HC CDR2
AIDPETGNTAYNQKFKG

SEQ ID NO: 8 - protein, 6 aa, artificial
  D1.2 HC CDR3
SRGFDY

SEQ ID NO: 9 - protein, 451 aa, artificial
  D1.2 VH
QVQLQQSGAELVRPGASVTLSCKASGNTFTDYEIHWVKQTPVHGLEWIGAIDPETGNTAYNQKFKGKARLTADKSSSTAYMELRSLTSEDSAVYYCTRSRGFDYWGQGTTLTVSSAKTTPPSVYPLAPGCGDTTGSSVTLGCLVKGYFPESVTVTWNSGSLSSSVHTFPALLQSGLYTMSSSVTVPSSTWPSQTVTCSVAHPASSTTVDKKLEPSGPISTINPCPPCKECHKCPAPNLEGGPSVFIFPPNIKDVLMISLTPKVTCVVVDVSEDDPDVRISWFVNNVEVHTAQTQTHREDYNSTIRVVSALPIQHQDWMSGKEFKCKVNNKDLPSPIERTISKIKGLVRAPQVYILPPPAEQLSRKDVSLTCLVVGFNPGDISVEWTSNGHTEENYKDTAPVLDSDGSYFIYSKLDIKTSKWEKTDSFSCNVRHEGLKNYYLKKTISRSPGK

SEQ ID NO: 10 - protein, 11 aa, Artificial
  C10.2 LC CDR1
QASQGTSINLN

SEQ ID NO: 11 - protein, 7 aa, artificial
  C10.2 LC CDR2
GASNLED

SEQ ID NO: 12 - protein, 7 aa, artificial
  C10.2 LC CDR3
LQHTYLP

SEQ ID NO: 13 - protein, 214 aa, artificial
  C10.2 VL
DVQMIQSPSSLSASLGDIVTMTCQASQGTSINLNWFQQKPGKAPKLLIYGASNLEDGVPSRFSGSRYGTDFTLTISSLEDEDMATYFCLQHTYLPFTFGSGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 14 - protein, 13 aa, artificial
  C10.2 HC CDR1
KASGYTFTDRTIH

SEQ ID NO: 15 - protein, 17 aa, artificial
  C10.2 HC CDR2
YIYPGDGSTKYNENFKG

SEQ ID NO: 16 - protein, 6 aa, artificial
  C10.2 HC CDR3
RGAMDY

SEQ ID NO: 17 - protein, 439 aa, artificial
  C10.2 VH
QVQLQQSDAELVKPGASVKISCKASGYTFTDRTIHWVKQRPEQGLEWIGYIYPGDGSTKYNENFKGKATLTADKSSSTAYMQLNSLTSEDSAVYFCARRGAMDYWGQGTSVTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 18 - protein, 11 aa, artificial
  C5.2 LC CDR1
QASQDTSINLN

SEQ ID NO: 19 - protein, 7 aa, artificial
  C5.2 LC CDR2
GASNLED

SEQ ID NO: 20 - protein, 7 aa, artificial
  C5.2 LC CDR3
LQHTYLP

SEQ ID NO: 21 - protein, 214 aa, artificial
  C5.2 VL
DVQMIQSPSSLSASLGDIVTMTCQASQDTSINLNWFQQKPGKAPKLLIYGASNLEDGVPSRFSGSRYGTDFTLTISSLEDEDMATYFCLQHTYLPFTFGSGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 22 - protein, 13 aa, artificial
  C5.2 HC CDR1
KASGYTFTDRTIH

SEQ ID NO: 23 - protein, 17 aa, artificial
  C5.2 HC CDR2
YIYPGDDSTKYNDNFKG

SEQ ID NO: 24 - protein, 6 aa, artificial
  C5.2 HC CDR3
RGTMDY

SEQ ID NO: 25 - protein, 439 aa, artificial
  C5.2 VH
QVQLQQSDAELVKPGASVKISCKASGYTFTDRTIHWVKQRPEQGLEWIGYIYPGDDSTKYNDMFKAKATLTADKSSNTAYMQLNSLTSDDSAVYFCARRGTMDYWGQGTSVTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 26 - protein, 11 aa, artificial
  C8.3 LC CDR1
QASQGTSINLN

SEQ ID NO: 27 - protein, 7 aa, artificial
  C8.3 LC CDR2
GSSNLED

SEQ ID NO: 28 - protein, 7 aa, artificial
  C8.3 LC CDR3
LQHSYLP

SEQ ID NO: 29 - protein, 214 aa, artificial
  C8.3 VL
DVQMIQSPSSLSASLGDIVTMTCQASQGTSINLNWFQQKPGKAPKLLIYGSSNLEDGVPSRFSGSRYGTDFTLTISSLEDEDMATYFCLQHSYLPFTFGSGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 30 - protein, 13 aa, artificial
  C8.3 HC CDR1
KASGYTFTDRTIH

SEQ ID NO: 31 - protein, 17 aa, artificial
  C8.3 HC CDR2
YIYPGDGSTKYNENFKG

SEQ ID NO: 32 - protein, 6 aa, artificial
  C8.3 HC CDR3
RGAMDY

SEQ ID NO: 33 - protein, 439 aa, artificial
  C8.3 VH
QVQLQQSDAELVNPGASVKISCKASGYTFTDRTIHWVKQRPEQGLEWIGYIYPGDGSTKYNENFKGKATLTADKSSSTAYMQLNSLASEDSAVYFCARRGAMDYWGQGTSVTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 34 - protein, 11 aa, artificial
  C10-2 Humanized LC CDR1
QASQDTSINLN

SEQ ID NO: 35 - protein, 7 aa, artificial
  C10-2 Humanized LC CDR2
GASNLET

SEQ ID NO: 36 - protein, 9 aa, artificial
  C10-2 Humanized LC CDR3
LQHTYLPFT

SEQ ID NO: 37 - protein, 214 aa, Artificial
  C10-2 Humanized VL
DVQMTQSPSSLSASVGDRVTMTCQASQDTSINLNWFQQKPGKAPKLLIYGASNLETGVPSRFSGSRSGTDFTLTISSLQPEDMATYYCLQHTYLPFTFGSGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 38 - protein, 5 aa, artificial
  C10-2 Humanized HC CDR1
DRTIH

SEQ ID NO: 39 - protein, 17 aa, artificial
  C10-2 Humanized HC CDR2
YIYPGDGSTKYSQKFQG

SEQ ID NO: 40 - protein, 6 aa, artificial
  C10-2 Humanized HC CDR3
RGAMDY

SEQ ID NO: 41 - protein, 445 aa, artificial
  C10-2 Humanized VH
QVQLVQSGAEVVKPGASVKISCKASGYTFTDRTIHWVRQAPGQGLEWIGYIYPGDGSTKYSQKFQGRATLTADTSASTAYMELSSLRSEDTAVYYCARRGAMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 42 - protein, 18 aa, artificial
  D55E HC CDR2
YIYPGEGSTKYSQKFQGR

SEQ ID NO: 43 - protein, 444 aa, artificial
  D55E VH
QVQLVQSGAEVVKPGASVKISCKASGYTFTDRTIHWVRQAPGQGLEWIGYIYPGEGSTKYSQKFQGRATLTADTSASTAYMELSSLRSEDTAVYYCARRGAMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 44 - protein, 18 aa, artificial
  D55Q HC CDR2
YIYPGQGSTKYSQKFQGR

SEQ ID NO: 45 - protein, 444 aa, artificial
  D55Q VH
QVQLVQSGAEVVKPGASVKISCKASGYTFTDRTIHWVRQAPGQGLEWIGYIYPGQGSTKYSQKFQGRATLTADTSASTAYMELSSLRSEDTAVYYCARRGAMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 46 - protein, 18 aa, artificial
  D55S HC CDR2
YIYPGSGSTKYSQKFQGR

SEQ ID NO: 47 - protein, 444 aa, Artificial
  D55S VH
QVQLVQSGAEVVKPGASVKISCKASGYTFTDRTIHWVRQAPGQGLEWIGYIYPGSGSTKYSQKFQGRATLTADTSASTAYMELSSLRSEDTAVYYCARRGAMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 48 - protein, 11 aa, artificial
  N32S LC CDR1
QASQDTSISLN

SEQ ID NO: 49 - protein, 214 aa, artificial
  N32S VL
DVQMTQSPSSLSASVGDRVTMTCQASQDTSISLNWFQQKPGKAPKLLIYGASNLETGVPSRFSGSRSGTDFTLTISSLQPEDMATYYCLQHTYLPFTFGSGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 50 - protein, 11 aa, artificial
  N32Q LC CDR1
QASQDTSIQLQ

SEQ ID NO: 51 - protein, 214 aa, artificial
  N32Q VH
DVQMTQSPSSLSASVGDRVTMTCQASQDTSIQLNWFQQKPGKAPKLLIYGASNLETGVPSRFSGSRSGTDFTLTISSLQPEDMATYYCLQHTYLPFTFGSGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 52 - protein, 11 aa, artificial
  N34S LC CDR1
QASQDTSINLS

SEQ ID NO: 53 - protein, 214 aa, Artificial
  N34S VL
DVQMTQSPSSLSASVGDRVTMTCQASQDTSINLSWFQQKPGKAPKLLIYGASNLETGVPSRFSGSRSGTDFTLTISSLQPEDMATYYCLQHTYLPFTFGSGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 54 - protein, 11 aa, artificial
  N34Q LC CDR1
QASQDTSINLQ

SEQ ID NO: 55 - protein, 214 aa, artificial
  N34Q VL
DVQMTQSPSSLSASVGDRVTMTCQASQDTSINLQWFQQKPGKAPKLLIYGASNLETGVPSRFSGSRSGTDFTLTISSLQPEDMATYYCLQHTYLPFTFGSGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 56 - protein, 11 aa, artificial
  N32S,N34S LC CDR1
QASQDTSISLS

SEQ ID NO: 57 - protein, 214 aa, artificial
  N32S,N34S VL
DVQMTQSPSSLSASVGDRVTMTCQASQDTSISLSWFQQKPGKAPKLLIYGASNLETGVPSRFSGSRSGTDFTLTISSLQPEDMATYYCLQHTYLPFTFGSGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 58 - protein, 11 aa, Artificial
  N32Q, N34S LC CDR1
QASQDTSIQLS

SEQ ID NO: 59 - protein, 214 aa, artificial
  N32Q, N34S VL
DVQMTQSPSSLSASVGDRVTMTCQASQDTSIQLSWFQQKPGKAPKLLIYGASNLETGVPSRFSGSRSGTDFTLTISSLQPEDMATYYCLQHTYLPFTFGSGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 60 - protein, 11 aa, artificial
  N32Q, N34Q LC CDR1
QASQDTSIQLQ

SEQ ID NO: 61 - protein, 214 aa, artificial
  N32Q, N34Q VL
DVQMTQSPSSLSASVGDRVTMTCQASQDTSIQLQWFQQKPGKAPKLLIYGASNLETGVPSRFSGSRSGTDFTLTISSLQPEDMATYYCLQHTYLPFTFGSGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 62 - protein, 11 aa, artificial
  N32S, N34Q LC CDR1
QASQDTSISLQ

SEQ ID NO: 63 - protein, 214 aa, artificial
  N32S, N34Q LC
DVQMTQSPSSLSASVGDRVTMTCQASQDTSISLQWFQQKPGKAPKLLIYGASNLETGVPSRFSGSRSGTDFTLTISSLQPEDMATYYCLQHTYLPFTFGSGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 64 - protein, 6 aa, artificial
  A101 HC CDR3
RGTMDY

SEQ ID NO: 65 - protein, 444 aa, artificial
  A101 VH
QVQLVQSGAEVVKPGASVKISCKASGYTFTDRTIHWVRQAPGQGLEWIGYIYPGDGSTKYSQKFQGRATLTADTSASTAYMELSSLRSEDTAVYYCARRGTMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 66 - protein, 18 aa, artificial
  D55E, A101T HC CDR2
YIYPGQGSTKYSQKFQGR

SEQ ID NO: 67 - protein, 6 aa, artificial
  D55E, A101T HC CDR3
RGTMDY

SEQ ID NO: 68 - protein, 444 aa, artificial
  D55E, A101T VH
QVQLVQSGAEVVKPGASVKISCKASGYTFTDRTIHWVRQAPGQGLEWIGYIYPGEGSTKYSQKFQGRATLTADTSASTAYMELSSLRSEDTAVYYCARRGTMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 69 - protein, 18 aa, artificial
  D55Q, A101T HC CDR2
YIYPGQGSTKYSQKFQGR

SEQ ID NO: 70 - protein, 6 aa, artificial
  D55Q, A101T HC CDR3
RGTMDY

SEQ ID NO: 71 - protein, 444 aa, artificial
  D55Q, A101T VH
QVQLVQSGAEVVKPGASVKISCKASGYTFTDRTIHWVRQAPGQGLEWIGYIYPGQGSTKYSQKFQGRATLTADTSASTAYMELSSLRSEDTAVYYCARRGTMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 72 - protein, 18 aa, artificial
  D55S, A101T HC CDR2
YIYPGSGSTKYSQKFQGR

SEQ ID NO: 73 - protein, 6 aa, artificial
  D55S, A101T HC CDR3
RGTMDY

SEQ ID NO: 74 - protein, 444 aa, artificial
  D55S, A101T VH
QVQLVQSGAEVVKPGASVKISCKASGYTFTDRTIHWVRQAPGQGLEWIGYIYPGSGSTKYSQKFQGRATLTADTSASTAYMELSSLRSEDTAVYYCARRGTMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

(19) United States
(12) Patent Application Publication
(10) Pub. No.: US 2022/0187322 A1
(43) Pub. Date: Jun. 16, 2022

(54) USE OF A PS396 ASSAY TO DIAGNOSE TAUOPHATIES

(71) Applicant: H. Lundbeck A/S, Valby (DK)

(72) Inventors: Jan Torleif Pedersen, Valby (DK); Thomas Karikari, Gothenburg (SE); Kina Höglund, Gothenburg (SE); Kaj Blennow, Gothenburg (SE); Mikkel Harndahl Harndahl, Valby (DK); Henrik Zetterberg, Gothenburg (SE)

(73) Assignee: H. Lundbeck A/S, Valby (DK)

(21) Appl. No.: 17/598,837

(22) PCT Filed: Mar. 24, 2020
(86) PCT No.: PCT/EP2020/058062

§ 371 Date: Sep. 27, 2021

(30) Foreign Application Priority Data
Mar. 28, 2019 (DK) PA201900377

(51) Int. Cl. G01N 33/68; G01N 33/543; G01N 1/40

(52) U.S. Cl. CPC G01N 33/6896; G01N 2800/2814; G01N 1/40; G01N 33/54326

(57) ABSTRACT

The present invention relates to an in vitro assay for measuring phosphorylated tau in a sample, said assay comprises the use of 2 antibodies i) a capture antibody specific for pS396 on tau and ii) a detection antibody binding tau on a different epitope that the capture antibody.

The present invention relates to the use of anti-tau antibodies in an assay to differentiate tau species in different tau pathologies. The assays according to the invention can be used i.e. to diagnose patients with tauopathies such as Alzheimer's disease, Pick's disease, corticobasal degeneration, progressive supranuclear palsy and globular glial tauopathy.

### BACKGROUND OF THE INVENTION

The term tauopathy defines a group of pathological diseases characterized by deposition of the microtubule-associated protein tau. The deposited tau is phosphorylated abnormally and accumulates as intracellular inclusions. There are a number of specific tauopathies, each of which vary by the distribution and morphological appearances of the protein-containing inclusions, as well as the relative burden of pathology affecting neurons and neuronal processes versus glial and glial processes (Dickson et al., 2011). The most common tauopathies are progressive supranuclear palsy (PSP), corticobasal degeneration (CBD), Pick's disease, and globular glial tauphathy (GGT) and chronic traumatic encephalopathy (CTE). All but Pick's disease are commonly associated with movement disorders (Keith A. Josephs, Chapter IX, in Movement Disorders (Second Edition), 2015). Tauopathies are also denoted Fronto Temporal Dementia (FTD), Fronto Temporal Lobar Degeneration (FTLD). Most cases of FTD are associated with genetic mutations in MAPT (tau) or GRN (granulin). FTD is associated with accumulation of tau protein.

In order to diagnose and find the right treatment for these patients it is important to have a method that can diagnose these patients and differentiate tau pathology that is characteristic for each disease. The inventors of the present invention have provided a number of assays that can help in the diagnosis of these diseases.

### SUMMARY OF THE INVENTION

The present invention relates to an in vitro assay for measuring phosphorylated tau in a sample, said assay comprises the use of 2 antibodies i) a capture antibody specific for the phosphorylated(p) serine(S) residue 396 (pS396) on tau and ii) a detection antibody binding tau on a different epitope than the capture antibody. The detector antibody may bind a non-phosphorylated residue on tau as disclosed further herein.

In a second aspect the invention relates to a method for measuring phosphorylated tau in a sample, which method comprises the steps of
a. Mixing capture antibodies specific for pS396 attached to paramagnetic beads with biotinylated detection antibodies and a sample,
b. Incubating the mixture at a sufficient time to allow the antibodies to bind to tau in the sample (e.g. 1, 2, 3 or 5 minutes or more),
c. Optionally, washing the mixture in step b) after incubation,
d. Adding streptavidin-conjugated beta-galactosidase and allowing said streptavidin-conjugate and the biotinylated detector antibody to react (e.g. 1, 2, 3 or 5 minutes or more),
e. Optionally washing the obtained mixture in step d),
f. Adding resorufin beta-D-galactopyranoside to the mixture in step d) or e) and allowing the hydrolysis of resorufin beta-D-galactopyranoside (e.g. 1, 2, 3 or 5 minutes or more),
g. Reading the fluorescence signal and comparing the signal with a standard

### FIGURE

FIG. 1 Schematic illustration of the pS396 tau assays.

(A) The Tau12-pS396 assay, also known as the full-length pS396 (or FL pS396) assay, uses the anti-pS396 antibody as the capture antibody and Tau12 (epitope at amino acids 6-18) as the detection antibody.
(B) The HT7-pS396 (mid-region pS396 or MR pS396) assay measures pS396 on tau species that stretch from the mid-region (epitope 159-168). This assay uses the pS396 antibody as the capture and HT7 the detection antibody.
(C) The pS396-Tau46 (C-terminus pS396 or CT pS396) assay, which uses the anti-pS396 antibody as capture and Tau46 (epitope 404-441) as detection antibody, is specific for pS396 phosphorylated tau that contains the extreme C-terminus region (amino acids 404-441).

FIG. 2 pS396 phosphorylated tau species differentiate neurodegenerative diseases with tau pathology. TBS-soluble fractions of frontal grey matter brain isolates from individuals with clinically confirmed tauopathies were tested with the FL, MR and CT pS396 assays. Five hundred-fold dilutions of equimolar concentrations (0.46 mg/ml) of each specimen (n=24 total) prepared with the assay diluent were analysed with each pS396 assay. Dilution corrected data (mean±standard error of the mean [SEM]) have been shown here. The samples consisted of Alzheimer's disease (AD, n=5), Pick's disease (PiD, n=5), corticobasal degeneration (CBD, n=5), progressive supranuclear palsy (PSP, n=5), globular glial tauopathy (GGT, n=2), and healthy controls (Ctrl, n=2 [n=1 for the MR assay]).
(A) Concentration of FL pS396 in the tauopathy brain samples. Mean concentrations±SEM: AD=5769±621 pg/ml, PiD=4737±960 pg/ml, CBD=10716±2452 pg/ml, PSP=8888±1002 pg/ml, GGT=10122±3955 pg/ml, Ctrl=3326±73 pg/ml. No statistically significant difference was recorded between the groups (Kruskal-Wallis test followed by Dunn's multiple comparison test).
(B) MR pS396 concentrations in brain specimen from different tauopathies. Mean levels±SEM: AD=23432±4773 pg/ml, PiD=29949±6938 pg/ml, CBD=17434±9359 pg/ml, PSP=5563±1047 pg/ml, GGT=9830±3933 pg/ml, Ctrl=1862 pg/ml. No statistically significant difference was recorded between the groups (one-way Analysis of variance [ANOVA]).
(C) Levels of CT pS396 in brain samples from five different tauopathy groups compared to controls. Mean concentration±SEM: AD=17692±2060 pg/ml, PiD=12549±1701 pg/ml, CBD=11203±5698 pg/ml, PSP=4766±706 pg/ml, GGT=7857±3097 pg/ml, Ctrl=397±54 pg/ml. CT pS396 concentrations in AD were significantly higher compared to same in PSP (p<0.05) and Ctrl (p<0.01). Similarly, CT pS396 levels in PiD were significantly higher than same in controls (Ctrls) (p<0.05; Kruskal-Wallis test followed by Dunn's multiple comparison test).
(D) The ratio of MR to FL pS396 levels significantly separated the different tauopathies. The MR/FL ratio for AD was significantly different compared to those in PiD, CBD, PSP, GGT and controls (p<0.01 each). Moreover, the ratio of MR to FL in the PiD patients was significantly different from CBD, PSP, GGT and controls (p=0.0001 each; one-way ANOVA followed by the Dunnett's multiple comparison test).
(E) Significant difference in the ratio of CT to FL pS396 concentrations in the disease groups. The CT/FL pS396 levels were significantly different in AD compared to CBD, PSP, GGT and controls (p<0.01 each), and PiD compared to CBD, PSP, GGT and controls (p<0.01 each; one-way ANOVA followed by the Dunnett's multiple comparison test).

All three assays measure tau pS396 in human tauopathy brain samples. The results indicate that the populations of pS396 tau in different tauopathies significantly vary with respect to the tau species present, suggesting that the combined measurement of pS396 and tau fragmentation is a promising approach to separating between these diseases.

FIG. 3 High levels of pS396-Tau46 (CT pS396) in the rTg4510 transgenic tau mouse model of Alzheimer's disease. (A) CSF samples from different animals were analysed with the CT pS396 assay at 50 or 75 fold dilutions. The measured and dilution-adjusted concentrations are both shown. (B) CT pS396 concentrations in 10 or 20 fold dilutions of plasma samples from six different animals, including those whose CSF levels of CT pS396 are shown in (A). Assay Limit of detection=1.50 pg/ml

CT pS396 is present in the CSF and plasma of rTg4510 transgenic tau mouse animals. The CT pS396 assay is an important tool for studying molecular changes in CT pS396 processing in this model and for preclinical evaluation of drug efficacy.

FIG. 4 Measurement of CT pS396 in human CSF. CT pS396 signal in 15 ml human CSF was enriched by spin filtration (using the Ultracel®-YM3 device; conditions shown in Table 1) followed by size exclusion chromatography (SEC) with the S200 10/300 GL column in 50 mM Tris pH 7.5, 10% glycerol running buffer. The eluted fractions were analysed directly with the Simoa CT pS396.
(A) Elution profile of the retentate of spin-filtered human CSF showing elution volume on the horizontal axis and UV absorbance on the vertical axis. The gridlines show the elution volumes of molecular weight markers: blue dextran (void volume, 2000 kDa; 7.76 ml), albumin (66 kDa; 13.45 ml), carbonic anhydrase (29 kDa; 16.28 ml), cytochrome (12.4 kDa; 20.39 ml), aprotinin (6.5 kDa; 24.36 ml).
(B) Chromatogram of spin-concentrated human CSF, showing the elution fraction IDs on the horizontal axis and UV absorbance on the vertical axis. Gridlines refer to the same molecular weight markers shown in (A).
(C) Concentration of CT pS396 in neat (untreated) CSF, spin-filtered CSF (retentate), and the eluted SEC fractions. No CT pS396 signal was detected in the neat CSF, which explains the need for pre-processing to enrich the signal. CT pS396 was not detected in the filtration product either. However, CT pS396 could be measured after SEC fractionation of the filtration product. The highest concentrations of CT pS396 were found in fractions C1, C2 and C3 (6.1, 9.9, and 6.1 pg/ml respectively in this sample) corresponding to elution volumes 12.0-13.5 ml and molecular weight ˜66 kDa. These properties suggest that the fractions eluting at 12.0-13.5 ml are enriched in tau monomers containing both the pS396 and Tau46 epitopes.

CT pS396 is not measurable in untreated human CSF. However, pre-analytical processing by spin filtration and SEC enriches CT pS396 signal, with the highest concentrations eluting at 12.0-13.5 ml. Consistent results have been recorded using spin filters of different capacities and models (Table 1), by changing the CSF starting volume (Table 1), and by varying the SEC elution volumes collected per fraction.

The sequential treatment of CSF samples by spin filtration followed by SEC fractionation is necessary for the CT pS396 signal enrichment because omitting either step leads to much reduced or undetectable amounts.

### DETAILED DESCRIPTION OF THE INVENTION

Traditional ELISA has been used for many years in analysing molecules of different kinds in samples. The present invention is directed to the use of the single molecule array assay (Simoa) ELISA technology to detect tau species in samples from patients diagnosed with tauopathies such as Alzheimer's disease, Pick's disease, corticobasal degeneration, progressive supranuclear palsy, globular glial tauopathy and chronic traumatic encephalopathy

In the present invention tau is human tau of the following sequence, Methionine being number 1

(SEQ ID NO.: 1)
MAEPRQEFEVMEDHAGTYGLGDRKDQGGYTMHQDQEGDTDAGLKESPLQT
PTEDGSEEPGSETSDAKSTPTAEDVTAPLVDEGAPGKQAAAQPHTEIPEG
TTAEEAGIGDTPSLEDEAAGHVTQARMVSKSKDGTGSDDKKAKGADGKTK
IATPRGAAPPGQKGQANATRIPAKTPPAPKTPPSSGEPPKSGDRSGYSSP
GSPGTPGSRSRTPSLPTPPTREPKKVAVVRTPPKSPSSAKSRLQTAPVPM
PDLKNVKSKIGSTENLKHQPGGGKVQIINKKLDLSNVQSKCGSKDNIKHV
PGGGSVQIVYKPVDLSKVTSKCGSLGNIHHKPGGGQVEVKSEKLDFKDRV
QSKIGSLDNITHVPGGGNKKIETHKLTFRENAKAKTDHGAEIVYKSPVVS
GDTSPRHLSNVSSTGSIDMVDSPQLATLADEVSASLAKQGL

In the first step of the single-molecule immunoassay capture antibodies are attached to the surface of paramagnetic beads (˜2.7 um diameter) that will be used to concentrate a dilute solution of tau molecules in a sample. A biotinylated detection antibody is added to the mixture, and the capture and detector antibodies are allowed to react to the tau in the sample. To remove non-specific protein binding the mixture may be washed. Subsequently beta-galactosidase-labelled streptavidin is added, followed by an optional washing step, and resorufin beta-D-galctopyranoside is added. The reaction mixture is allowed to react and generate a fluorescent product which may be read and analysed in an appropriate machine.

The assay developed by the inventors of the present invention is based on two antibodies, a capture antibody and a biotinylated detector antibody. The capture antibody is conjugated to a paramagnetic bead as described in Example 1. These are specific for the phosphorylated (p) S396 of tau. The generation of such antibodies are disclosed in for example WO2017/009308 and these antibodies are further described in Table I below. In the Examples of the present invention the pS396 specific antibody used is the antibody designated “010-2 Humanized” from patent WO2018/011073 and described in Table II below

TABLE I
pS396 antibodies disclosed in WO2017/009308
D1.2

| | CDR1 | CDR2 | CDR3 |
|---|---|---|---|
| Light Chain | (SEQ ID NO.: 2) | (SEQ ID NO.: 3) | (SEQ ID NO.: 4) |
| | RSSQSLVHSN GNTYLH | KVSNRFS | SQSTHVP |
| VL | DVMMTQTPLS LPVSLGDQAS ISCRSSQSLV HSNGNTYLHW HLQKPGQSPK | | |
| (SEQ ID NO.: 5) | FLIYKVSNRF | | |
| | SGVPDRFSGS GSGTDFTLKI SRVEAEDLGV YFCSQSTHVP FTFGSGTKLE | | |
| | IKRADAAPTV | | |
| | SIFPPSSEQL TSGGASVVCF LNNFYPKDIN VKWKIDGSER QNGVLNSWTD | | |
| | QDSKDSTYSM | | |
| | SSTLTLTKDE YERHNSYTCE ATHKTSTSPI VKSFNRNEC | | |
| | CDR1 | CDR2 | CDR3 |
| Heavy Chain | (SEQ ID NO.: 6) | (SEQ ID NO.: 7) | (SEQ ID NO.: 8) |
| | KASGNTFTDY EIH | AIDPETGNTA | SRGFDY |
| | YNQKFKG | | |

| | |
|---|---|
| VH | QVQLQQSGAE LVRPGASVTL SCKASGNTFT DYEIHWVKQT PVHGLEWIGA |
| (SEQ ID NO.: 9) | IDPETGNTAY |
| | NQKFKGKARL TADKSSSTAY MELRSLTSED SAVYYCTRSR GFDYWGQGTT |
| | LTVSSAKTTP |
| | PSVYPLAPGC GDTTGSSVTL GCLVKGYFPE SVTVTWNSGS LSSSVHTFPA |
| | LLQSGLYTMS |
| | SSVTVPSSTW PSQTVTCSVA HPASSTTVDK KLEPSGPIST INPCPPCKEC |
| | HKCPAPNLEG |
| | GPSVFIFPPN IKDVLMISLT PKVTCVVVDV SEDDPDVRIS WFVNNVEVHT |
| | AQTQTHREDY |
| | NSTIRVVSAL PIQHQDWMSG KEFKCKVNNK DLPSPIERTI SKIKGLVRAP |
| | QVYILPPPAE |
| | QLSRKDVSLT CLVVGFNPGD ISVEWTSNGH TEENYKDTAP VLDSDGSYFI |
| | YSKLDIKTSK |
| | WEKTDSFSCN VRHEGLKNYY LKKTISRSPG K |

C10.2

| | CDR1 | CDR2 | CDR3 |
|---|---|---|---|
| Light Chain | (SEQ ID NO.: 10) | (SEQ ID NO.: 11) | (SEQ ID NO.: 12) |
| | QASQGTSINL N | GASNLED | LQHTYLP |
| VL | DVQMIQSPSS LSASLGDIVT MTCQASQGTS INLNWFQQKP GKAPKLLIYG | | |
| (SEQ ID NO.: 13) | ASNLEDGVPS | | |
| | RFSGSRYGTD FTLTISSLED EDMATYFCLQ HTYLPFTFGS GTKLEIKRAD | | |
| | AAPTVSIFPP | | |
| | SSEQLTSGGA SVVCFLNNFY PKDINVKWKI DGSERQNGVL NSWTDQDSKD | | |
| | STYSMSSTLT | | |
| | LTKDEYERHN SYTCEATHKT STSPIVKSFN RNEC | | |
| | CDR1 | CDR2 | CDR3 |
| Heavy Chain | (SEQ ID NO.: 14) | (SEQ ID NO.: 15) | (SEQ ID NO.: 16) |
| | KASGYTFTDR TIH | YIYPGDGSTK | RGAMDY |
| | | YNENFKG | |

| | |
|---|---|
| VH | QVQLQQSDAE LVKPGASVKI SCKASGYTFT DRTIHWVKQR PEQGLEWIGY |
| (SEQ ID NO.: 17) | IYPGDGSTKY |
| | NENFKGKATL TADKSSSTAY MQLNSLTSED SAVYFCARRG AMDYWGQGTS |
| | VTVSSAKTTP |
| | PSVYPLAPGS AAQTNSMVTL GCLVKGYFPE PVTVTWNSGS LSSGVHTFPA |
| | VLQSDLYTLS |
| | SSVTVPSSTW PSETVTCNVA HPASSTKVDK KIVPRDCGCK PCICTVPEVS |
| | SVFIFPPKPK |
| | DVLTITLTPK VTCVVVDISK DDPEVQFSWF VDDVEVHTAQ TQPREEQFNS |
| | TFRSVSELPI |
| | MHQDWLNGKE FKCRVNSAAF PAPIEKTISK TKGRPKAPQV YTIPPPKEQM |
| | AKDKVSLTCM |
| | ITDFFPEDIT VEWQWNGQPA ENYKNTQPIM DTDGSYFVYS KLNVQKSNWE |
| | AGNTFTCSVL |
| | HEGLHNHHTE KSLSHSPGK |

C5.2

| | CDR1 | CDR2 | CDR3 |
|---|---|---|---|
| Light Chain | (SEQ ID NO.: 18) | (SEQ ID NO.: 19) | (SEQ ID NO.: 20) |
| | QASQDTSINL N | GASNLED | LQHTYLP |
| VL | DVQMIQSPSS LSASLGDIVT MTCQASQDTS INLNWFQQKP GKAPKLLIYG | | |
| (SEQ ID NO.: 21) | ASNLEDGVPS | | |
| | RFSGSRYGTD FTLTISSLED EDMATYFCLQ HTYLPFTFGS GTKLEIKRAD | | |
| | AAPTVSIFPP | | |
| | SSEQLTSGGA SVVCFLNNFY PKDINVKWKI DGSERQNGVL NSWTDQDSKD | | |
| | STYSMSSTLT LTKDEYERHN SYTCEATHKT STSPIVKSFN RNEC | | |
| | CDR1 | CDR2 | CDR3 |
| Heavy Chain | (SEQ ID NO.: 22) | (SEQ ID NO.: 23) | (SEQ ID NO.: 24) |
| | KASGYTFTDR TIH | YIYPGDDSTK | RGTMDY |
| | | YNDNFKG | |

| | |
|---|---|
| VH | QVQLQQSDAE LVKPGASVKI SCKASGYTFT DRTIHWVKQR PEQGLEWIGY |
| (SEQ ID NO.: 25) | IYPGDDSTKY |
| | NDMFKAKATL TADKSSNTAY MQLNSLTSDD SAVYFCARRG TMDYWGQGTS |
| | VTVSSAKTTP |
| | PSVYPLAPGS AAQTNSMVTL GCLVKGYFPE PVTVTWNSGS LSSGVHTFPA |
| | VLQSDLYTLS |
| | SSVTVPSSTW PSETVTCNVA HPASSTKVDK KIVPRDCGCK PCICTVPEVS |
| | SVFIFPPKPK |
| | DVLTITLTPK VTCVVVDISK DDPEVQFSWF VDDVEVHTAQ TQPREEQFNS |
| | TFRSVSELPI |
| | MHQDWLNGKE FKCRVNSAAF PAPIEKTISK TKGRPKAPQV YTIPPPKEQM |
| | AKDKVSLTCM |
| | ITDFFPEDIT VEWQWNGQPA ENYKNTQPIM DTDGSYFVYS KLNVQKSNWE |
| | AGNTFTCSVL |
| | HEGLHNHHTE KSLSHSPGK |

C8.3

| | CDR1 | CDR2 | CDR3 |
|---|---|---|---|
| Light Chain | (SEQ ID NO.: 26) | (SEQ ID NO.: 27) | (SEQ ID NO.: 28) |
| | QASQGTSINL N | GSSNLED | LQHSYLP |
| VL | DVQMIQSPSS LSASLGDIVT MTCQASQGTS INLNWFQQKP GKAPKLLIYG | | |
| (SEQ ID NO.: 29) | SSNLEDGVPS | | |
| | RFSGSRYGTD FTLTISSLED EDMATYFCLQ HSYLPFTFGS GTKLEIKRAD | | |
| | AAPTVSIFPP | | |
| | SSEQLTSGGA SVVCFLNNFY PKDINVKWKI DGSERQNGVL NSWTDQDSKD | | |
| | STYSMSSTLT 180 | | |
| | LTKDEYERHN SYTCEATHKT STSPIVKSFN RNEC | | |
| | CDR1 | CDR2 | CDR3 |
| Heavy Chain | (SEQ ID NO.: 30) | (SEQ ID NO.: 31) | (SEQ ID NO.: 32) |
| | KASGYTFTDR TIH | YIYPGDGSTK | RGAMDY |
| | | YNENFKG | |

| | |
|---|---|
| VH | QVQLQQSDAE LVNPGASVKI SCKASGYTFT DRTIHWVKQR PEQGLEWIGY |
| (SEQ ID NO.: 33) | IYPGDGSTKY |
| | NENFKGKATL TADKSSSTAY MQLNSLASED SAVYFCARRG AMDYWGQGTS |
| | VTVSSAKTTP |
| | PSVYPLAPGS AAQTNSMVTL GCLVKGYFPE PVTVTWNSGS LSSGVHTFPA |
| | VLQSDLYTLS |
| | SSVTVPSSTW PSETVTCNVA HPASSTKVDK KIVPRDCGCK PCICTVPEVS |
| | SVFIFPPKPK |
| | DVLTITLTPK VTCVVVDISK DDPEVQFSWF VDDVEVHTAQ TQPREEQFNS |
| | TFRSVSELPI |
| | MHQDWLNGKE FKCRVNSAAF PAPIEKTISK TKGRPKAPQV YTIPPPKEQM |
| | AKDKVSLTCM |
| | ITDFFPEDIT VEWQWNGQPA ENYKNTQPIM DTDGSYFVYS KLNVQKSNWE |
| | AGNTFTCSVL |
| | HEGLHNHHTE KSLSHSPGK |

Table II: pS396 Antibodies Disclosed in WO2018/011073

The antibodies in the below scheme are all engineered versions of the antibody designated “010-2 humanized antibody”. Differences compared to the 010-2 humanized antibody are shown specifically, otherwise grey boxes in the table are intended to indicate identical amino acids as the 010-2 humanized antibody. Thus, for example, D55E has the same CDR 1-3 of the light chain and CDR1 and 3 of the heavy chain as the 010-2 humanized antibody (grey boxes), whereas the CDR2 of the heavy chain differs (amino acid residues are given) and thus VH differs from the 010-2 humanized antibody (amino acid residues are given)

C10-2 Humanized

| | CDR1 | CDR2 | CDR3 |
|---|---|---|---|
| Light Chain | (SEQ ID NO.: 34) | (SEQ ID NO.: 35) | (SEQ ID NO.: 36) |
| | QASQDTSINL N | GASNLET | LQHTYLPFT |
| VL | DVQMTQSPSS LSASVGDRVT MTCQASQDTS INLNWFQQKP GKAPKLLIYG | | |
| (SEQ ID NO.: 37) | ASNLETGVPS | | |
| | RFSGSRSGTD FTLTISSLQP EDMATYYCLQ HTYLPFTFGS GTKLEIKRTV | | |
| | AAPSVFIFPP | | |
| | SDEQLKSGTA SVVCLLNNFY PREAKVQWKV DNALQSGNSQ ESVTEQDSKD | | |
| | STYSLSSTLT | | |
| | LSKADYEKHK VYACEVTHQG LSSPVTKSFN RGEC | | |
| | CDR1 | CDR2 | CDR3 |
| Heavy Chain | (SEQ ID NO.: 38) | (SEQ ID NO.: 39) | (SEQ ID NO.: 40) |
| | DRTIH | YIYPGDGSTK | RGAMDY |
| | | YSQKFQG | |

| | |
|---|---|
| VH | QVQLVQSGAE VVKPGASVKI SCKASGYTFT DRTIHWVRQA PGQGLEWIGY |
| (SEQ ID NO.: 41) | IYPGDGSTKY |
| | SQKFQGRATL TADTSASTAY MELSSLRSED TAVYYCARRG AMDYWGQGTS |
| | VTVSSASTKG |
| | PSVFPLAPSS KSTSGGTAAL GCLVKDYFPE PVTVSWNSGA LTSGVHTFPA |
| | VLQSSGLYSL |
| | SSVVTVPSSS LGTQTYICNV NHKPSNTKVD KRVEPKSCDK THTCPPCPAP |
| | ELLGGPSVFL |
| | FPPKPKDTLM ISRTPEVTCV VVDVSHEDPE VKFNWYVDGV EVHNAKTKPR |
| | EEQYNSTYRV |
| | VSVLTVLHQD WLNGKEYKCK VSNKALPAPI EKTISKAKGQ PREPQVYTLP |
| | PSREEMTKNQ |
| | VSLTCLVKGF YPSDIAVEWE SNGQPENNYK TTPPVLDSDG SFFLYSKLTV |
| | DKSRWQQGNV |
| | FSCSVMHEAL HNHYTQKSLS LSPGK |

D55E

| Light Chain | CDR1 | CDR2 | CDR3 |
|---|---|---|---|
| VL | | | |
| | | CDR2 | |
| Heavy Chain | CDR1 | (SEQ ID NO.: 42) | CDR3 |
| | | YIYPGEGSTK | |
| | | YSQKFQGR | |

| | |
|---|---|
| VH | QVQLVQSGAE VVKPGASVKI SCKASGYTFT DRTIHWVRQA PGQGLEWIGY |
| (SEQ ID NO.: 43) | IYPGEGSTKY |
| | SQKFQGRATL TADTSASTAY MELSSLRSED TAVYYCARRG AMDYWGQGTS |
| | VTVSSASTKG |
| | PSVFPLAPSS KSTSGGTAAL GCLVKDYFPE PVTVSWNSGA LTSGVHTFPA |
| | VLQSSGLYSL |
| | SSVVTVPSSS LGTQTYICNV NHKPSNTKVD KRVEPKSCDK THTCPPCPAP |
| | ELLGGPSVFL |
| | FPPKPKDTLM ISRTPEVTCV VVDVSHEDPE VKFNWYVDGV EVHNAKTKPR |
| | EEQYNSTYRV |
| | VSVLTVLHQD WLNGKEYKCK VSNKALPAPI EKTISKAKGQ PREPQVYTLP |
| | PSREEMTKNQ |
| | VSLTCLVKGF YPSDIAVEWE SNGQPENNYK TTPPVLDSDG SFFLYSKLTV |
| | DKSRWQQGNV |
| | FSCSVMHEAL HNHYTQKSLS LSPG |

D55Q

| Light Chain | CDR1 | CDR2 | CDR3 |
|---|---|---|---|
| VL | | | |
| | | CDR2 | |
| Heavy Chain | CDR1 | (SEQ ID NO.: 44) | CDR3 |
| | | YIYPGQGSTK | |
| | | YSQKFQGR | |

| | |
|---|---|
| VH | QVQLVQSGAE VVKPGASVKI SCKASGYTFT DRTIHWVRQA PGQGLEWIGY |
| (SEQ ID NO.: 45) | IYPGQGSTKY |
| | SQKFQGRATL TADTSASTAY MELSSLRSED TAVYYCARRG AMDYWGQGTS |
| | VTVSSASTKG |
| | PSVFPLAPSS KSTSGGTAAL GCLVKDYFPE PVTVSWNSGA LTSGVHTFPA |
| | VLQSSGLYSL |
| | SSVVTVPSSS LGTQTYICNV NHKPSNTKVD KRVEPKSCDK THTCPPCPAP |
| | ELLGGPSVFL |
| | FPPKPKDTLM ISRTPEVTCV VVDVSHEDPE VKFNWYVDGV EVHNAKTKPR |
| | EEQYNSTYRV |
| | VSVLTVLHQD WLNGKEYKCK VSNKALPAPI EKTISKAKGQ PREPQVYTLP |
| | PSREEMTKNQ |
| | VSLTCLVKGF YPSDIAVEWE SNGQPENNYK TTPPVLDSDG SFFLYSKLTV |
| | DKSRWQQGNV |
| | FSCSVMHEAL HNHYTQKSLS LSPG |

D55S

| Light Chain | CDR1 | CDR2 | CDR3 |
|---|---|---|---|
| VL | | | |
| | | CDR2 | |
| Heavy Chain | CDR1 | (SEQ ID NO.: 46) | CDR3 |
| | | YIYPGSGSTK | |
| | | YSQKFQGR | |

| | |
|---|---|
| VH | QVQLVQSGAE VVKPGASVKI SCKASGYTFT DRTIHWVRQA PGQGLEWIGY |
| (SEQ ID NO.: 47) | IYPGSGSTKY |
| | SQKFQGRATL TADTSASTAY MELSSLRSED TAVYYCARRG AMDYWGQGTS |
| | VTVSSASTKG |
| | PSVFPLAPSS KSTSGGTAAL GCLVKDYFPE PVTVSWNSGA LTSGVHTFPA |
| | VLQSSGLYSL |
| | SSVVTVPSSS LGTQTYICNV NHKPSNTKVD KRVEPKSCDK THTCPPCPAP |
| | ELLGGPSVFL |
| | FPPKPKDTLM ISRTPEVTCV VVDVSHEDPE VKFNWYVDGV EVHNAKTKPR |
| | EEQYNSTYRV |
| | VSVLTVLHQD WLNGKEYKCK VSNKALPAPI EKTISKAKGQ PREPQVYTLP |
| | PSREEMTKNQ |
| | VSLTCLVKGF YPSDIAVEWE SNGQPENNYK TTPPVLDSDG SFFLYSKLTV |
| | DKSRWQQGNV |
| | FSCSVMHEAL HNHYTQKSLS LSPG |

N32S

| | CDR1 | | |
|---|---|---|---|
| Light Chain | (SEQ ID NO.: 48) | CDR2 | CDR3 |
| | QASQDTSISL N | | |
| VL | DVQMTQSPSS LSASVGDRVT MTCQASQDTS ISLNWFQQKP GKAPKLLIYG | | |
| (SEQ ID NO.: 49) | ASNLETGVPS | | |
| | RFSGSRSGTD FTLTISSLQP EDMATYYCLQ HTYLPFTFGS GTKLEIKRTV | | |
| | AAPSVFIFPP | | |
| | SDEQLKSGTA SVVCLLNNFY PREAKVQWKV DNALQSGNSQ ESVTEQDSKD | | |
| | STYSLSSTLT | | |
| | LSKADYEKHK VYACEVTHQG LSSPVTKSFN RGEC | | |
| Heavy Chain | CDR1 | CDR2 | CDR3 |
| VH | | | |

N32Q

| | CDR1 | | |
|---|---|---|---|
| Light Chain | (SEQ ID NO.: 50) | CDR2 | CDR3 |
| | QASQDTSIQL Q | | |
| VL | DVQMTQSPSS LSASVGDRVT MTCQASQDTS IQLNWFQQKP GKAPKLLIYG | | |
| (SEQ ID NO.: 51) | ASNLETGVPS | | |
| | RFSGSRSGTD FTLTISSLQP EDMATYYCLQ HTYLPFTFGS GTKLEIKRTV | | |
| | AAPSVFIFPP | | |
| | SDEQLKSGTA SVVCLLNNFY PREAKVQWKV DNALQSGNSQ ESVTEQDSKD | | |
| | STYSLSSTLT | | |
| | LSKADYEKHK VYACEVTHQG LSSPVTKSFN RGEC | | |
| Heavy Chain | CDR1 | CDR2 | CDR3 |
| VH | | | |

N34S

| | CDR1 | | |
|---|---|---|---|
| Light Chain | (SEQ ID NO.: 52) | CDR2 | CDR3 |
| | QASQDTSINL S | | |
| VL | DVQMTQSPSS LSASVGDRVT MTCQASQDTS INLSWFQQKP GKAPKLLIYG | | |
| (SEQ ID NO.: 53) | ASNLETGVPS | | |
| | RFSGSRSGTD FTLTISSLQP EDMATYYCLQ HTYLPFTFGS GTKLEIKRTV | | |
| | AAPSVFIFPP | | |
| | SDEQLKSGTA SVVCLLNNFY PREAKVQWKV DNALQSGNSQ ESVTEQDSKD | | |
| | STYSLSSTLT | | |
| | LSKADYEKHK VYACEVTHQG LSSPVTKSFN RGEC | | |
| Heavy Chain | CDR1 | CDR2 | CDR3 |
| VH | | | |

N34Q

| | CDR1 | | |
|---|---|---|---|
| Light Chain | (SEQ ID NO.: 54) | CDR2 | CDR3 |
| | QASQDTSINL Q | | |
| VL | DVQMTQSPSS LSASVGDRVT MTCQASQDTS INLQWFQQKP GKAPKLLIYG | | |
| (SEQ ID NO.: 55) | ASNLETGVPS | | |
| | RFSGSRSGTD FTLTISSLQP EDMATYYCLQ HTYLPFTFGS GTKLEIKRTV | | |
| | AAPSVFIFPP | | |
| | SDEQLKSGTA SVVCLLNNFY PREAKVQWKV DNALQSGNSQ ESVTEQDSKD | | |
| | STYSLSSTLT | | |
| | LSKADYEKHK VYACEVTHQG LSSPVTKSFN RGEC | | |
| Heavy Chain | CDR1 | CDR2 | CDR3 |
| VH | | | |

N32S, N34S

| | CDR1 | | |
|---|---|---|---|
| Light Chain | (SEQ ID NO.: 56) | CDR2 | CDR3 |
| | QASQDTSISL S | | |
| VL | DVQMTQSPSS LSASVGDRVT MTCQASQDTS ISLSWFQQKP GKAPKLLIYG | | |
| (SEQ ID NO.: 57) | ASNLETGVPS | | |
| | RFSGSRSGTD FTLTISSLQP EDMATYYCLQ HTYLPFTFGS GTKLEIKRTV | | |
| | AAPSVFIFPP | | |
| | SDEQLKSGTA SVVCLLNNFY PREAKVQWKV DNALQSGNSQ ESVTEQDSKD | | |
| | STYSLSSTLT | | |
| | LSKADYEKHK VYACEVTHQG LSSPVTKSFN RGEC | | |
| Heavy Chain | CDR1 | CDR2 | CDR3 |
| VH | | | |

N32Q, N34S

| | CDR1 | | |
|---|---|---|---|
| Light Chain | (SEQ ID NO.: 58) | CDR2 | CDR3 |
| | QASQDTSIQL S | | |
| VL | DVQMTQSPSS LSASVGDRVT MTCQASQDTS IQLSWFQQKP GKAPKLLIYG | | |
| (SEQ ID NO.: 59) | ASNLETGVPS | | |
| | RFSGSRSGTD FTLTISSLQP EDMATYYCLQ HTYLPFTFGS GTKLEIKRTV | | |
| | AAPSVFIFPP | | |
| | SDEQLKSGTA SVVCLLNNFY PREAKVQWKV DNALQSGNSQ ESVTEQDSKD | | |
| | STYSLSSTLT LSKADYEKHK VYACEVTHQG LSSPVTKSFN RGEC | | |
| Heavy Chain | CDR1 | CDR2 | CDR3 |
| VH | | | |

N32Q, N34Q

| | CDR1 | | |
|---|---|---|---|
| Light Chain | (SEQ ID NO.: 60) | CDR2 | CDR3 |
| | QASQDTSIQL Q | | |
| VL | DVQMTQSPSS LSASVGDRVT MTCQASQDTS IQLQWFQQKP GKAPKLLIYG | | |
| (SEQ ID NO.: 61) | ASNLETGVPS | | |
| | RFSGSRSGTD FTLTISSLQP EDMATYYCLQ HTYLPFTFGS GTKLEIKRTV | | |
| | AAPSVFIFPP | | |
| | SDEQLKSGTA SVVCLLNNFY PREAKVQWKV DNALQSGNSQ ESVTEQDSKD | | |
| | STYSLSSTLT | | |
| | LSKADYEKHK VYACEVTHQG LSSPVTKSFN RGEC | | |
| Heavy Chain | CDR1 | CDR2 | CDR3 |
| VH | | | |

N32S, N34Q

| | CDR1 | | |
|---|---|---|---|
| Light Chain | (SEQ ID NO.: 62) | CDR2 | CDR3 |
| | QASQDTSISL Q | | |
| VL | DVQMTQSPSS LSASVGDRVT MTCQASQDTS ISLQWFQQKP GKAPKLLIYG | | |
| (SEQ ID NO.: 63) | ASNLETGVPS | | |
| | RFSGSRSGTD FTLTISSLQP EDMATYYCLQ HTYLPFTFGS GTKLEIKRTV | | |
| | AAPSVFIFPP | | |
| | SDEQLKSGTA SVVCLLNNFY PREAKVQWKV DNALQSGNSQ ESVTEQDSKD | | |
| | STYSLSSTLT | | |
| | LSKADYEKHK VYACEVTHQG LSSPVTKSFN RGEC | | |
| Heavy Chain | CDR1 | CDR2 | CDR3 |
| VH | | | |

A101

| Light Chain | CDR1 | CDR2 | CDR3 |
|---|---|---|---|
| VL | | | |
| | | | CDR3 |
| Heavy Chain | CDR1 | CDR2 | (SEQ ID NO.: 64) |
| | | | RGTMDY |

| | |
|---|---|
| VH | QVQLVQSGAE VVKPGASVKI SCKASGYTFT DRTIHWVRQA PGQGLEWIGY |
| (SEQ ID NO.: 65) | IYPGDGSTKY |
| | SQKFQGRATL TADTSASTAY MELSSLRSED TAVYYCARRG TMDYWGQGTS |
| | VTVSSASTKG |
| | PSVFPLAPSS KSTSGGTAAL GCLVKDYFPE PVTVSWNSGA LTSGVHTFPA |
| | VLQSSGLYSL |
| | SSVVTVPSSS LGTQTYICNV NHKPSNTKVD KRVEPKSCDK THTCPPCPAP |
| | ELLGGPSVFL |
| | FPPKPKDTLM ISRTPEVTCV VVDVSHEDPE VKFNWYVDGV EVHNAKTKPR |
| | EEQYNSTYRV |
| | VSVLTVLHQD WLNGKEYKCK VSNKALPAPI EKTISKAKGQ PREPQVYTLP |
| | PSREEMTKNQ |
| | VSLTCLVKGF YPSDIAVEWE SNGQPENNYK TTPPVLDSDG SFFLYSKLTV |
| | DKSRWQQGNV |
| | FSCSVMHEAL HNHYTQKSLS LSPG |

D55E, A101T

| Light Chain | CDR1 | CDR2 | CDR3 |
|---|---|---|---|
| VL | | | |
| | | CDR2 | CDR3 |
| Heavy Chain | CDR1 | (SEQ ID NO.: 66) | (SEQ ID NO.: 67) |
| | | YIYPGQGSTK | RGTMDY |
| | | YSQKFQGR | |

| | |
|---|---|
| VH | QVQLVQSGAE VVKPGASVKI SCKASGYTFT DRTIHWVRQA PGQGLEWIGY |
| (SEQ ID NO.: 68) | IYPGEGSTKY |
| | SQKFQGRATL TADTSASTAY MELSSLRSED TAVYYCARRG TMDYWGQGTS |
| | VTVSSASTKG |
| | PSVFPLAPSS KSTSGGTAAL GCLVKDYFPE PVTVSWNSGA LTSGVHTFPA |
| | VLQSSGLYSL |
| | SSVVTVPSSS LGTQTYICNV NHKPSNTKVD KRVEPKSCDK THTCPPCPAP |
| | ELLGGPSVFL |
| | FPPKPKDTLM ISRTPEVTCV VVDVSHEDPE VKFNWYVDGV EVHNAKTKPR |
| | EEQYNSTYRV |
| | VSVLTVLHQD WLNGKEYKCK VSNKALPAPI EKTISKAKGQ PREPQVYTLP |
| | PSREEMTKNQ |
| | VSLTCLVKGF YPSDIAVEWE SNGQPENNYK TTPPVLDSDG SFFLYSKLTV |
| | DKSRWQQGNV |
| | FSCSVMHEAL HNHYTQKSLS LSPG |

D55Q, A101T

| Light Chain | CDR1 | CDR2 | CDR3 |
|---|---|---|---|
| VL | | | |
| | | CDR2 | CDR3 |
| Heavy Chain | CDR1 | (SEQ ID NO.: 69) | (SEQ ID NO.: 70) |
| | | YIYPGQGSTK | RGTMDY |
| | | YSQKFQGR | |

| | |
|---|---|
| VH | QVQLVQSGAE VVKPGASVKI SCKASGYTFT DRTIHWVRQA PGQGLEWIGY |
| (SEQ ID NO.: 71) | IYPGQGSTKY |
| | SQKFQGRATL TADTSASTAY MELSSLRSED TAVYYCARRG TMDYWGQGTS |
| | VTVSSASTKG |
| | PSVFPLAPSS KSTSGGTAAL GCLVKDYFPE PVTVSWNSGA LTSGVHTFPA |
| | VLQSSGLYSL |
| | SSVVTVPSSS LGTQTYICNV NHKPSNTKVD KRVEPKSCDK THTCPPCPAP |
| | ELLGGPSVFL |
| | FPPKPKDTLM ISRTPEVTCV VVDVSHEDPE VKFNWYVDGV EVHNAKTKPR |
| | EEQYNSTYRV |
| | VSVLTVLHQD WLNGKEYKCK VSNKALPAPI EKTISKAKGQ PREPQVYTLP |
| | PSREEMTKNQ |
| | VSLTCLVKGF YPSDIAVEWE SNGQPENNYK TTPPVLDSDG SFFLYSKLTV |
| | DKSRWQQGNV |
| | FSCSVMHEAL HNHYTQKSLS LSPG |

D55S, A101T

| Light Chain | CDR1 | CDR2 | CDR3 |
|---|---|---|---|
| VL | | | |
| | | CDR2 | CDR3 |
| Heavy Chain | CDR1 | (SEQ ID NO.: 72) | (SEQ ID NO.: 73) |
| | | YIYPGSGSTK | RGTMDY |
| | | YSQKFQGR | |

| | |
|---|---|
| VH | QVQLVQSGAE VVKPGASVKI SCKASGYTFT DRTIHWVRQA PGQGLEWIGY |
| (SEQ ID NO.: 74) | IYPGSGSTKY |
| | SQKFQGRATL TADTSASTAY MELSSLRSED TAVYYCARRG TMDYWGQGTS |
| | VTVSSASTKG |
| | PSVFPLAPSS KSTSGGTAAL GCLVKDYFPE PVTVSWNSGA LTSGVHTFPA |
| | VLQSSGLYSL |
| | SSVVTVPSSS LGTQTYICNV NHKPSNTKVD KRVEPKSCDK THTCPPCPAP |
| | ELLGGPSVFL |
| | FPPKPKDTLM ISRTPEVTCV VVDVSHEDPE VKFNWYVDGV EVHNAKTKPR |
| | EEQYNSTYRV |
| | VSVLTVLHQD WLNGKEYKCK VSNKALPAPI EKTISKAKGQ PREPQVYTLP |
| | PSREEMTKNQ |
| | VSLTCLVKGF YPSDIAVEWE SNGQPENNYK TTPPVLDSDG SFFLYSKLTV |
| | DKSRWQQGNV |
| | FSCSVMHEAL HNHYTQKSLS LSPG |

N32S, A101T

| Light Chain | CDR1 | CDR2 | CDR3 |
|---|---|---|---|
| | Same sequence as | | |
| | N32S CDR1 | | |
| VL | Same VL sequence as N32S | | |
| Heavy Chain | CDR1 | CDR2 | CDR3 |
| | | | Same sequence as |
| | | | A101 CDR3 |

| | |
|---|---|
| VH | Same VH sequence as A101 |

N32Q, A101T

| Light Chain | CDR1 | CDR2 | CDR3 |
|---|---|---|---|
| | Same sequence as | | |
| | N32Q CDR1 | | |
| VL | Same VL sequence as N32Q | | |
| Heavy Chain | CDR1 | CDR2 | CDR3 |
| | | | Same sequence as |
| | | | A101 CDR3 |

| | |
|---|---|
| VH | Same VH sequence as A101 |

N34S, A101T

| Light Chain | CDR1 | CDR2 | CDR3 |
|---|---|---|---|
| | Same sequence as | | |
| | N34s CDR1 | | |
| VL | Same VL sequence as N34S | | |
| Heavy Chain | CDR1 | CDR2 | CDR3 |
| | | | Same sequence as |
| | | | A101 CDR3 |

| | |
|---|---|
| VH | Same VH sequence as A101T |

N34Q, A101T

| Light Chain | CDR1 | CDR2 | CDR3 |
|---|---|---|---|
| | Same sequence as | | |
| | N34Q CDR1 | | |
| VL | Same VL sequence as N34Q | | |
| Heavy Chain | CDR1 | CDR2 | CDR3 |
| | | | Same sequence as |
| | | | A101 CDR3 |

| | |
|---|---|
| VH | Same VH sequence as A101 |

The detection/or detector (used interchangeably herein) antibodies used have been biotinylated as described in Example 2, and may bind the C-terminal, mid or N-terminal region of tau at a site different from the pS396 residue. The detector antibody may bind non-phosphorylated residues. In particular, epitopes of the C-terminus on tau are amino acids 1-20 (such as 6-18), the mid region of tau is amino acids 140-170 (such as 159-168) and N-terminus of tau is 400-441 (such as 404-421). In the Examples, the following antibodies are used:

Tau12 (#806502, BioLegend) is used and binds to the amino acids 6-18 of tau, detection antibody is HT7 (#MN1000, Invitrogen) binds mid region 159-168 amino acids, and Tau46 (#806601, BioLegend) binds the C-terminal region amino acids 404-441.

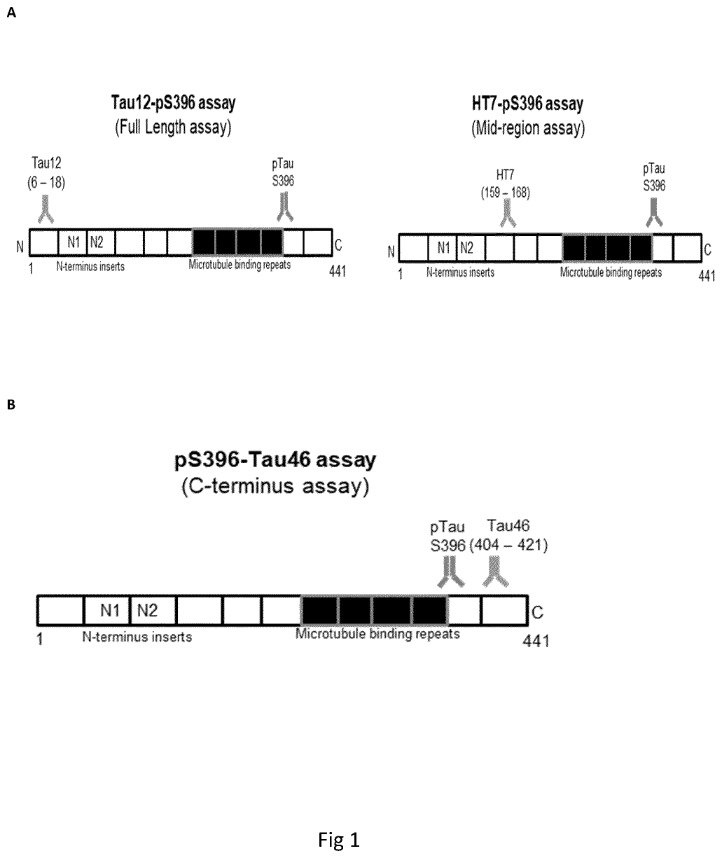

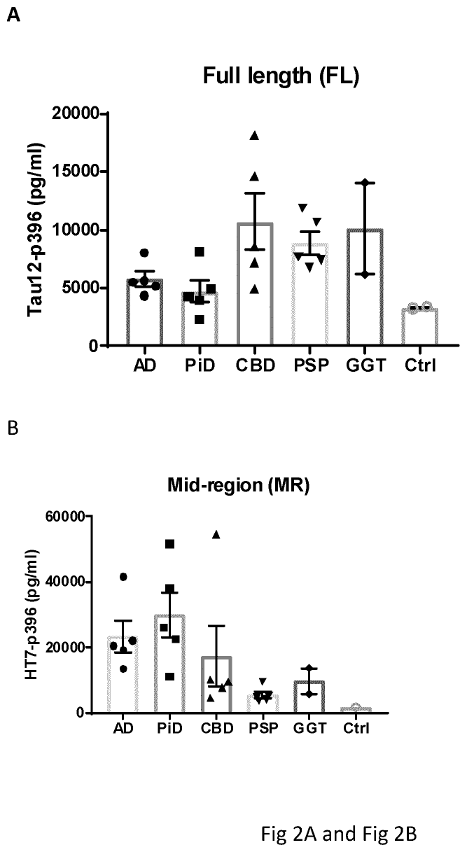

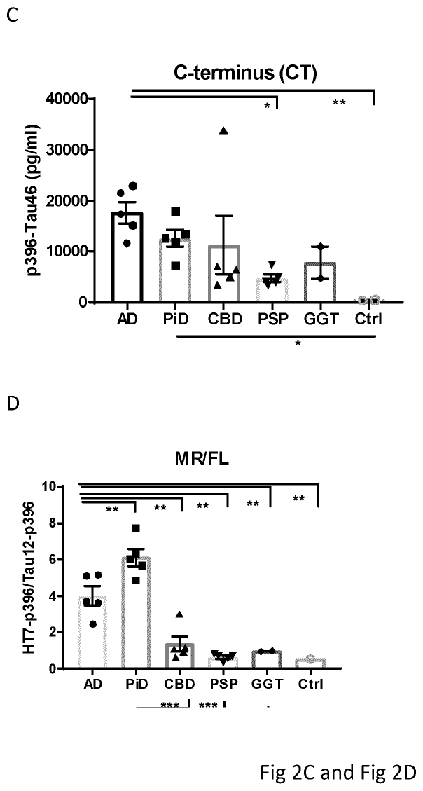

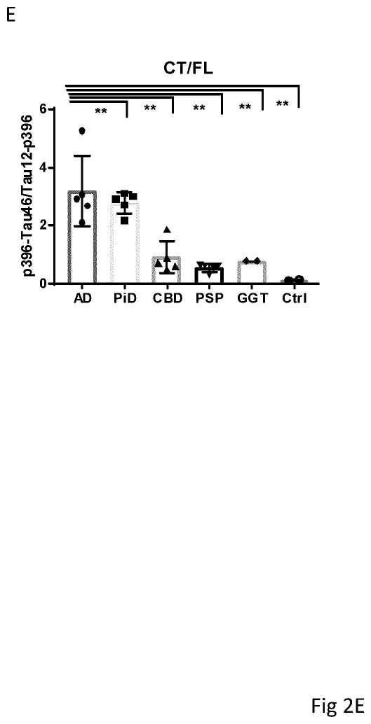

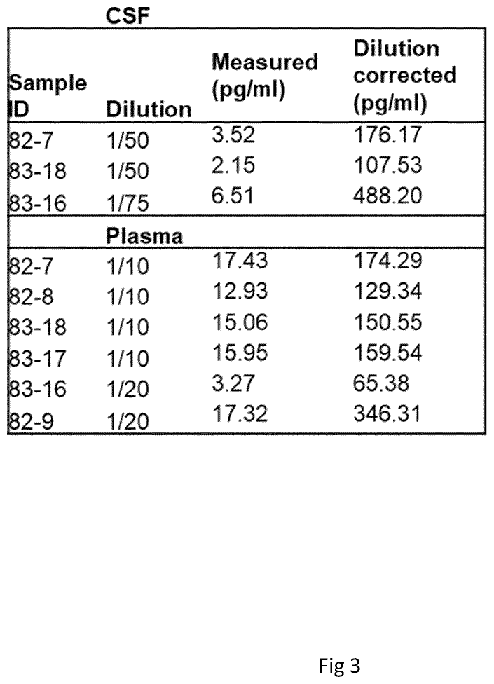

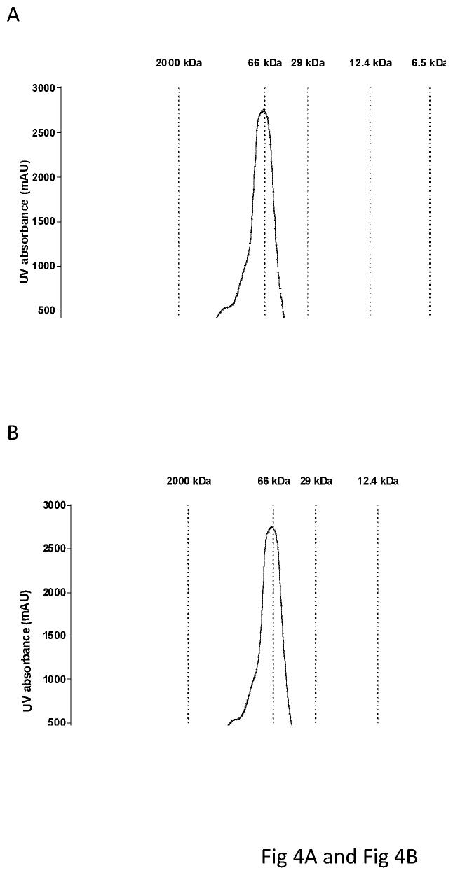

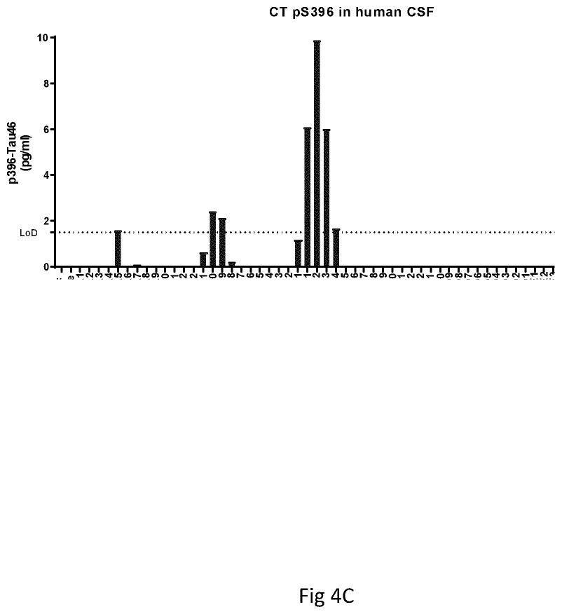

Three assays measuring pS396 on different tau species have been developed (FIG. 1); each uses the anti-pS396 capture antibody and share all other experimental conditions except the detection antibodies, which have been described below.
1. Full length pS396 (FL assay): this assay measures pS396 phosphorylated tau species stretching from the N-terminus region. The detection antibody, monoclonal Tau12 (#806502, BioLegend), binds to the amino acids 6-18 of tau.
2. Mid region pS396 (MR assay): this assay measures tau forms simultaneously carrying two epitopes: pS396 phosphorylation and the mid region 159-168 amino acids. The detection antibody is HT7 (#MN1000, Invitrogen).
3. C-terminus pS396 (CT assay): the assay is specific for pS396 phosphorylated tau that contains the extreme carboxyl terminus region (amino acids 404-441). The detection antibody is Tau46 (#806601, BioLegend).

The method, as described in Example 4, comprises the steps of
a. Mixing capture antibodies specific for pS396 attached to paramagnetic beads with a biotinylated detection antibody and a sample,
b. Incubating the mixture at a sufficient time to allow the antibodies to bind to tau in the sample (e.g. 1, 2, 3 or 5 minutes or more),
c. Optionally, washing the mixture in step b) after incubation,
d. Adding streptavidin-conjugated beta-galactosidase and allowing said streptavidin-conjugate and the biotinylated detector antibody to react (e.g. 1, 2, 3 or 5 minutes or more),
e. Optionally washing the obtained mixture after step d),
f. Adding resorufin beta-D-galactopyranoside to the mixture in step d) or e) and allowing the hydrolysis of resorufin beta-D-galactopyranoside (e.g. 1, 2, 3 or 5 minutes or more),
g. Reading the fluorescence signal and comparing the signal with a standard

The amount of pS396 conjugated antibody beads used are usually at least 1000 beads such as at least 10,000, 100,000 beads or more. The sample may be a CSF, plasma or bio-fluid sample from a mammal, for example a human CSF sample from a human suffering from a tauopathy such as Alzheimer's disease, Pick's disease, corticobasal degeneration, progressive supranuclear palsy or globular glial tauopathy. It may be advantageous to concentrate the sample with respect to tau for example by use of spin filtration columns and size exclusion chromatography as shown in Example 3.

The results of the assays used individually or in combination can be used to diagnose or differentiate tauopathies, such as Alzheimer's disease, Pick's disease, corticobasal degeneration, progressive supranuclear palsy and globular glial tauopathy, as shown in Example 4. For example, the CT assay can be used to diagnose Alzheimer's disease and Pick's disease. By comparing the MR/FL ratio it can be used to differentiate Alzheimer's disease over the other tauopathies and the control, and further the Pick's disease was significant different from corticobasal degeneration, progressive supranuclear palsy, globular glial tauopathy and the control. By suing the CT/FL ratio, Alzheimer's disease can be used to differentiate over the other tauopathies and the control and Pick's disease was different compared to corticobasal degeneration, progressive supranuclear palsy and globular glial tauopathy and control.

### EXPERIMENTAL DETAILS

### Example 1: Conjugation of Capture Antibody (pS396) to Paramagnetic Beads

The pS396 antibody was buffer exchanged into bead conjugation buffer (BCB; 50 mM MES pH 6.2) using Ultracel 50K spin filtration columns (#UFC505096, Amicon). The filter was first rinsed with 450 ul BCB by centrifuging at 14000×g at room temperature (RT) for 5 min, and discarding the flow-through. Thereafter, 1.6 g/L antibody was buffer exchanged into BCB by centrifuging at 14000×g, RT, for 5 min. The flow-through was discarded and the filter returned to the collection tube. BCB was added to the retentate to bring the volume to 450 ul, and re-centrifuged under the same conditions. This step was repeated once after discarding the flow-through. Subsequently, the filter was rinsed with 40 ul BCB, inverted into a new collection tube and the antibody recovered by centrifuging for 2 min at 1000 xg, RT. The concentration of the antibody was estimated with Nanodrop Lite (ThermoFisher Scientific) and stored at 4° C. until use.

Paramagnetic carboxylated singleplex beads (#103207, Quanterix) were washed thrice with bead wash buffer (BWB; 1×PBS+1% Tween 20) and then twice with BCB using a magnetic separator. The beads at a concentration of 1.4×10⁶ beads/μL were activated by adding 0.3 g/L 1-ethyl-3-(3-dimethylaminopropyl)carbodiimide hydrochloride (#A35391, Thermo Scientific) and incubating at 4° C. for 30 min. Thereafter, the activated beads were washed once with ice-cold BCB and the supernatant discarded. The antibody (0.2 g/L) was added to the beads and the mixture incubated for 2 h at RT with shaking to allow the antibodies bind to the beads. Shaking was always performed with a HulaMixer Sample Mixer (#15920D, ThermoFisher Scientific) under the following conditions with each step lasting 5 sec: orbital=5 rpm, reciprocal=90°, vibro/pause=5°. Afterwards, the supernatant was removed and the antibody-conjugated beads washed twice with BWB. The reaction was blocked with bead blocking buffer (1% BSA in 1×PBS) for 1 h at RT with shaking. Finally, the beads were washed twice with BWB and then once with bead diluent (BD; 50 mM Tris pH 7.8, 50 mM NaCl, 10 mM EDTA, 1% BSA, 0.1% Tween20). After removing the supernatant with a magnetic separator, the beads were resuspended in BD and stored at 4° C. until use.

### Example 2: Biotin Conjugation to Detection Antibody

The detection antibodies were buffer exchanged into biotinylation reaction buffer (BRB; 100 mM PBS pH 7.4) in Ultracel 50K spin filtration columns (#UFC505096, Amicon). After cleaning the column by centrifuging 450 ul BRB at 14000 xg, RT, for 5 min, the flow-through was discarded and the antibody transferred to the filter. BRB was added to bring the volume to 450 ul and centrifuged at 14000×g at RT for 5 min. The buffer exchange was repeated two more times, at each stage by bringing the antibody volume to 450 ul with BRB and centrifuging for 5 min at 14000 xg, RT. The filter was rinsed with 40 ul BRB, inverted into a new collection tube and the antibody recovered by centrifuging for 2 min at 1000 xg, RT. The concentration of the antibody was estimated using Nanodrop Lite. Forty times excess of EZ-Link NHS-PEG4-Biotin (#21329, Thermo Scientific) was added to the antibody and incubated for 30 min at RT. Free biotin was removed by repeating the buffer exchange process performed prior to the biotin labelling. The biotin-conjugated antibodies were stored at 4° C. until use.

### Example 3: Pre-Analytical Processing of Samples and Calibrators

Appropriate concentrations of the assay calibrator (recombinant tau 441 phosphorylated in vitro by Glycogen Synthase Kinase 3β (#T08-50FN, SignalChem)) were prepared by diluting stock concentrations with the assay diluent (Tau 2.0 diluent, #101556, Quanterix) before analysis. Quality control samples include TBS-soluble human Alzheimer's disease brain extract diluted 500 and 5000 times with the assay diluent.

Tris buffered saline (TBS)-soluble human brain extracts, rTg4510 transgenic mice CSF and plasma samples were diluted with Tau 2.0 diluent to the desired concentrations indicated in FIGS. 2 and 3 and their legends.

The level of pS396 tau in human CSF was enriched by concentrating samples in spin filtration columns and fractionating the retentate by size exclusion chromatography (SEC) on a Superdex S200 10/300 GL column (#17-5175-01, GE Healthcare) running on an Ethan LC system (GE Healthcare). The running buffer was 50 mM Tris pH 7.5+10% glycerol. Collected fractions were analysed directly using the Simoa pS396 assays. This method has been verified using spin filtration columns of different capacities and properties (Table 1).

TABLE 1
Details of centrifugal filter devices and conditions used to
concentrate human CSF prior to size-based fractionation
to enrich pS396 signal. All devices were
purchased from Merck Millipore.

| | Catalogue | Centrifugation | CSF |
|---|---|---|---|
| Filter name | number | conditions | volume |
| Ultracel ®- | 4203 | 6500 xg, 4 h, 4° C. | 12 × |
| YM3 | | | 2 ml* |
| Ultracel ®- | UFC900308 | 4000 xg, 40 min, | 15 ml |
| 3K | | 25° C. | |
| Centriprep | 4303 | 3000 xg, 95 min, 4° C. | 15 ml |
| Microcon ®- | MRCPRT010 | 14000 xg, 40 min, RT | 8 × |
| 10 | | | 0.5 ml* |
| Amicon ® | UFC200324 | 7500 xg, 40 min, RT | 2 ml |
| Ultra-2 ml | | | |

*Retentate fractions from the indicated number of columns were pooled for further analyses.

### Example 4: Single Molecule Array (Simoa) Assays

Each pS396 assay uses a two-step protocol on the Simoa HD-1 instrument (Quanterix, Lexington, Mass., USA). In this assay configuration, 100 ul of the bead mixture, consisting of 1000 beads/ul each of pS396 antibody-coated beads and Helper Beads (#103208, Quanterix), is aspirated into a reaction cuvette. Thereafter, 20 ul biotinylated detection antibody (2 ug/ml) and 100 ul of the analyte of interest were added and the reaction mixture incubated for 47 cadences (1 cadence=45 sec) to allow the analyte to react with the capture and detection antibodies. The beads were subsequently washed and 100 ul of 450 pM streptavidin-conjugated β-galactosidase (SBG; #100439, Quanterix). Following another incubation for 7 cadences and a subsequent wash, 25 μl resorufin β-D-galactopyranoside (RGP; #103159, Quanterix) was added. Hydrolysis of RGP was catalysed by SBG, yielding the fluorescent product resorufin. The beads were transferred onto a disc of 200,000 wells, each only large enough to accommodate one bead. Extra beads were removed and the disc surface sealed before imaging. The fluorescent signals were converted to average enzyme per bead (AEB) and the sample concentrations extrapolated from a four-parametric logistic calibration curve generated with known protein concentrations.

### Assay Setups

Three assays measuring pS396 on different tau species have been developed (FIG. 1); each uses the anti-pS396 capture antibody and share all other experimental conditions except the detection antibodies, which have been described below.
1. Full length pS396 (FL assay): this assay measures pS396 phosphorylated tau species stretching from the N-terminus region. The detection antibody, monoclonal Tau12 (#806502, BioLegend), binds to the amino acids 6-18 of tau.
2. Mid region pS396 (MR assay): this assay measures tau forms simultaneously carrying two epitopes: pS396 phosphorylation and the mid region 159-168 amino acids. The detection antibody is HT7 (#MN1000, Invitrogen).
3. C-terminus pS396 (CT assay): the assay is specific for pS396 phosphorylated tau that contains the extreme carboxyl terminus region (amino acids 404-441). The detection antibody is Tau46 (#806601, BioLegend).

1. An in vitro assay for measuring phosphorylated tau in a sample, said assay comprises the use of 2 antibodies i) a capture antibody specific for pS396 on tau and ii) a detection antibody binding tau on a different epitope than the capture antibody.

2. The assay according to claim 1, wherein said detection antibody is binding to an epitope within amino acids 1-20 on tau (such as 6-18), amino acids 140-170 (such as 159-168) on tau, or 400-441 (such as 404-421) on tau.

3. The assay according to claim 1, wherein the detection antibody is biotinylated.

4. The assay according to claim 1, wherein the capture antibody is attached to the surface of paramagnetic beads.

5. The assay according to claim 1, wherein beta-galactosidase conjugated streptavidin is used to generate a signal readout.

6. The assay according to claim 1, wherein the sample is a CSF, plasma or other bio-fluid sample from a mammal.

7. The assay according to claim 1, wherein the sample is a CSF or plasma sample from a human suffering from a tauopathy such as Alzheimer's disease, Pick's disease, corticobasal degeneration, progressive supranuclear palsy or globular glial tauopathy.

8. The assay according to claim 1, wherein the CSF sample has been concentrated with respect to tau for example by use of spin filtration columns and size exclusion chromatography.

9. (canceled)

10. A method for measuring phosphorylated tau in a sample, which method comprises the steps of
a. Mixing capture antibodies specific for pS396 attached to paramagnetic beads with biotinylated detection antibodies and a sample,
b. Incubating the mixture at a sufficient time to allow the antibodies to bind to tau in the sample (e.g. 1, 2, 3 or 5 minutes or more),
c. Optionally, washing the mixture in step b) after incubation,
d. Adding streptavidin-conjugated beta-galactosidase and allowing said streptavidin-conjugate and the biotinylated detector antibody to react (e.g. 1, 2, 3 or 5 minutes or more),
e. Optionally washing the obtained mixture after step d),
f. Adding resorufin beta-D-galactopyranoside to the mixture in step d) or e) and allowing the hydrolysis of resorufin beta-D-galactopyranoside (e.g. 1, 2, 3 or 5 minutes or more),
g. Reading the fluorescence signal and comparing the signal with a standard.

11. The method according to claim 10, wherein said capture antibodies are attached to paramagnetic beads and at least 1000 of said paramagnetic beads are added to the sample.

12. The method according to claim 10, wherein said detection antibody is binding to an epitope within amino acids 1-20 on tau (such as 6-18), amino acids 140-170 (such as 159-168) on tau, or 400-441 (such as 404-421) on tau.

13. The method according to claim 10, wherein the sample is a CSF, plasma or bio-fluid sample from a mammal.

14. The method according to claim 10, wherein the sample is a CSF or plasma sample from a human suffering from a tauopathy such as Alzheimer's disease, Pick's disease, corticobasal degeneration, progressive supranuclear palsy or globular glial tauopathy.

15. The method according to claim 10, wherein said sample has been concentrated with respect to tau for example by use of spin filtration columns and size exclusion chromatography.

16. A method for diagnosing a tauopathy such as Alzheimer's disease, Pick's disease, corticobasal degeneration, progressive supranuclear palsy or globular glial tauopathy,
comprising the method according to claim 10.

17. The method according to claim 10, wherein said detector antibody is binding an epitope within amino acids 400-441 (such as 404-421) on tau to diagnose Alzheimer's disease or Pick's disease.
